(19) United States
(12) Patent Application Publication
(10) Pub. No.: US 2026/0294684 A1
(43) Pub. Date: Oct. 1, 2026

(54) SYSTEMS AND METHODS FOR THERMAL MONITORING DURING PHACOEMULSIFICATION SURGERY

(71) Applicant: Johnson & Johnson Surgical Vision, Inc., Irvine, CA (US)

(72) Inventors: Deep Mehta, Irvine, CA (US); Ezra Spencer, Vista, CA (US)

(21) Appl. No.: 19/573,883

(22) Filed: Mar. 20, 2026

Related U.S. Application Data
(60) Provisional application No. 63/781,229, filed on Mar. 31, 2025.

(51) Int. Cl. A61F 9/007; A61M 1/00

(52) U.S. Cl. CPC A61F 9/00745; A61M 1/774; A61M 2205/18; A61M 2205/3368; A61M 2205/502; A61M 2210/0612

(57) ABSTRACT

A system and method for emulsification of particles produced during a surgical procedure is disclosed herein. Included are a surgical handpiece configured to emulsify lens material in an eye; an aspiration line coupled with the handpiece; and a surgical console communicatively coupled with the aspiration line and the surgical handpiece. The surgical console configured to estimate temperature of a tip of the surgical handpiece, and to modify at least one of ultrasound power to the surgical handpiece, a flow rate of the aspiration line, or a vacuum level of the aspiration line responsively to the estimated temperature meeting a predetermined temperature threshold.

### CROSS-REFERENCE TO RELATED APPLICATIONS

This application claims priority to U.S. Provisional Application No. 63/781,229, filed Mar. 31, 2025, the entire contents of which are hereby incorporated by reference in its entirety.

### FIELD OF THE DISCLOSURE

This disclosure relates generally to surgical handpieces, and more particularly to systems and methods for thermal monitoring of a surgical handpiece during phacoemulsification surgery.

### BACKGROUND

During certain types of surgeries, such as ophthalmic surgery, a surgical apparatus is used to perform surgical procedures. An ophthalmic surgical apparatus typically includes a handheld medical implement or tool, referred to as a handpiece, with a surgical tip and/or sleeve, and operating controls for regulating settings or functions associated with the handpiece or surgical tip. Control of the various operating settings or functions is based on the manner of use of the surgical tool. A control module may control power supply, an irrigation source, one or more aspiration pumps, and the associated electronic hardware and software for operating the surgical tool. The surgical tip may be ultrasonically driven once placed within a surgical incision to, for example, emulsify the cataractous lens of an eye. That is, the electrical energy received at the surgical tip converts to an emulsifying mechanical motion. During this emulsification, the eye may be irrigated, such as with a balanced salt solution, and the emulsified material may be aspirated from the eye.

More specifically, phacoemulsification includes making a corneal and/or scleral incision in the eye, and the insertion of the surgical tip (i.e., the distal end) of the handpiece through the incision. The surgical tip is then ultrasonically driven to emulsify the lens. The power supply to the ultrasonics is controlled to control the emulsification, and the control module also controls the irrigation source and the aspiration pump.

The surgical tip of the handpiece includes a needle and a sleeve that coaxially surrounds at least a portion of the needle. The sleeve has one or more irrigation ports proximate to the distal end and is coupled with the irrigation source via an irrigation input line. The needle includes an aspiration port at the distal tip and is coupled with the aspiration pump via an aspiration output line. Concomitantly with the emulsification, fluid from the irrigation source thus maintains positive pressure by gravity, and/or with external pressure source, in the eye. This positive pressure maintains the anterior chamber and capsular bag, and replenishes the fluid aspirated away with the emulsified crystalline lens material, which helps avoid undesirable damage to the patient's eye.

The irrigation fluid in the patient's eye and the crystalline lens material are aspirated from the eye by aspiration resulting from the aspiration pump, which may be a peristaltic or positive displacement pump. Other forms of aspiration pumps are well known in the art, e.g., Venturi pump, progressive cavity pump, etc. Alternatively, some procedures may include irrigating the eye and aspirating the irrigation fluid without concomitant destruction, alteration or removal of the lens.

Thus, phacoemulsification may involve combining irrigation, aspiration and emulsification within a single handpiece, and that handpiece is typically controlled electrically by a control module in order to, for example, control the flow of fluid through the handpiece and tip in a manner that avoids patient injury. Precise control over aspiration and irrigation to the ocular region is thus needed, which can be manual or automated.

As referenced, the control and settings of the system may be electronically controlled or modified by the control module and/or by a user/surgeon. The control module may also provide feedback information to a user or surgeon regarding the function and operation of the system, and may receive input from a user or surgeon in order to adjust surgical settings, particularly where aspects of the surgery are controlled, at least in part, manually. A user interface with a display system communicative with the control module is thus generally provided during use of the device.

Notwithstanding the safety features and controls of these manual and automated surgical techniques, damage to the patient, such as to that patient's eye, may still occur in rare circumstances. For example, as patient biology, surgical techniques, and cataract significance may vary from patient to patient in a phacoemulsification surgical context, patient injury, such as the occurrence of surgical wound burns, may also vary.

### BRIEF DESCRIPTION OF THE DRAWINGS

The following drawings are illustrative of particular examples of the present disclosure and therefore do not limit the scope of the present disclosure. The drawings are not to scale and are intended for use in conjunction with the explanations in the following detailed description. In the drawings, like reference numerals may represent like parts and assemblies throughout the several views.

FIG. 1 illustrates an exemplary phacoemulsification system;

FIG. 2 illustrates an exemplary graphical user interface;

FIG. 3 illustrates an exemplary graphical user interface;

FIG. 4 illustrates a flow diagram of exemplary aspects of the disclosure;

FIG. 5 illustrates a flow diagram of exemplary aspects of the disclosure;

FIG. 6 illustrates a flow diagram of exemplary aspects of the disclosure;

FIG. 7 illustrates a flow diagram of exemplary aspects of the disclosure;

FIGS. 8A and 8B are graphical illustrations of aspects of the disclosure;

FIGS. 9A and 9B are graphical illustrations of aspects of the disclosure;

FIGS. 10A and 10B are graphical illustrations of aspects of the disclosure;

FIGS. 11A and 11B are graphical illustrations of aspects of the disclosure;

FIGS. 12A and 12B are graphical illustrations of aspects of the disclosure; and

FIG. 13 is a graphical illustration of aspects of the disclosure.

### DETAILED DESCRIPTION

The following discussion omits or only briefly describes conventional features of surgical handpieces that are apparent to those skilled in the art. Those of ordinary skill may thus recognize that other elements may be desirable and/or necessary to implement the devices, systems, and methods described herein. It is noted that various examples are described in detail with reference to the drawings. Reference to these various examples does not limit the scope of the claims attached hereto. Additionally, any examples set forth in this specification are intended to be non-limiting and merely set forth some of the many possible implementations for the appended claims. Further, particular features described herein can be used in combination with other described features in each of the various possible combinations and permutations. As such, it is understood that the detailed description is exemplary and explanatory only and is not restrictive of the broad inventive concepts upon which the examples disclosed herein are based.

Unless otherwise specifically defined herein, all terms are to be given their broadest reasonable interpretation. This includes meanings implied from the specification as well as meanings understood by those skilled in the art and/or as defined in dictionaries, treatises, etc.

It is noted that, as used in the specification and the appended claims, the singular forms “a,” “an” and “the” include plural referents unless otherwise specified. The terms “includes” and/or “including,” when used in this specification, specify the presence of stated features, elements, and/or components, but do not preclude the presence or addition of one or more other features, steps, operations, elements, components, and/or groups thereof.

Relative terms such as “horizontal,” “vertical,” “up,” “down,” “top,” and “bottom” as well as derivatives thereof (e.g., “horizontally,” “downwardly,” “upwardly,” etc.) should be construed to refer to the orientation as then-described or as shown in the drawing figure under discussion. These relative terms are for convenience of description and normally are not intended to require a particular orientation in actuality. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The phrases “configured to”, “operatively” or “operably connected” indicates such an attachment, coupling or connection that allows the pertinent structures to operate as intended by virtue of that relationship.

Reference throughout the specification to “exemplary”, “one example”, “an example” or “some examples” means that a particular feature, structure, or characteristic is described in connection with at least one example of the subject matter disclosed. Thus, the appearance of the phrases “in one example”, “in an example”, “by way of example”, “in some examples”, and other like-phrases in various places throughout the specification do not necessarily refer to a single or the same example. Further, the particular features, structures or characteristics of “one example”, “an example”, “some examples”, or other like-phrases may be combined in any suitable manner with each other to form additional examples of such combinations. It is intended that examples of the disclosed subject matter cover modifications and variations thereof. Terms such as “first,” “second,” “third,” etc., merely identify one of a number of portions, components, steps, operations, functions, and/or points of reference as disclosed herein, and likewise do not necessarily limit examples of the present disclosure to any particular configuration or orientation.

The terms “proximal,” “distal,” “anterior,” “posterior,” “medial,” “lateral,” “superior,” and “inferior” are defined by their standard usage indicating a directional term of reference. For example, “proximal” refers to a position that is situated nearer to a body or point of attachment or interest, while “distal” refers to a position that is situated away from the body or point of attachment or interest. In another example, “anterior” refers to the front of a body or structure, while “posterior” refers to the rear of a body or structure, in relation to a relative viewpoint. In another example, “medial” refers to the direction towards the midline of a body or structure, and “lateral” refers to the direction away from the midline of a body or structure. In some examples, “lateral” or “laterally” may refer to any sideways direction. In another example, “superior” refers to the top of a body or structure, while “inferior” refers to the bottom of a body or structure. It should be understood, however, that the directional term of reference may be interpreted within the context of a specific body or structure, such that a directional term referring to a location in the context of the reference body or structure may remain consistent as the orientation of the body or structure changes.

Moreover, throughout this disclosure, various aspects may be presented in a range format. It should be understood that a description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range. In relation to a range and as used herein, the term “about” in reference to a measurable value, such as an amount, a temporal duration, and the like, is meant to encompass the specified value and/or variations of plus or minus 20%, plus or minus 10%, plus or minus 5%, plus or minus 1%, and plus or minus 0.1% of the specified value, as such variations are appropriate.

Processor-implemented modules and systems are disclosed herein that may provide access to and transformation of a plurality of types of non-transitory digital content, including but not limited to data streams, sensor output, and mathematical models, and the algorithms applied herein may track, deliver, manipulate, transform, transceive and report the accessed content. Described examples of these modules, apps, systems and methods are intended to be exemplary and not limiting.

Those of skill in the art would further appreciate that the various illustrative logical blocks, modules, circuits, and algorithm steps described in connection with the examples disclosed herein may be implemented as electronic hardware, computer software, or combinations of both. To clearly illustrate this interchangeability of hardware and software, various illustrative components, blocks, modules, circuits, and steps are described generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

An exemplary computing processing system for use in association with the examples, by way of non-limiting example, is capable of executing software, such as an operating system (OS), applications/apps, user interfaces, and/or one or more other computing algorithms, such as the algorithms, decisions, models, modules, programs and subprograms discussed herein. The operation of the exemplary processing system is controlled primarily by non-transitory computer readable instructions/code, such as instructions stored in a computer readable storage medium, such as hard disk drive (HDD), optical disk, solid state drive, Random Access Memory (RAM), a flash memory, or the like. Such instructions may be executed within the central processing unit (CPU) to cause the system to perform the disclosed operations.

In many known computer servers, workstations, mobile devices, personal computers, and the like, the CPU is implemented in an integrated circuit called a processor. It is appreciated that, although the exemplary processing system may comprise a single CPU, such description is merely illustrative, as the processing system may comprise a plurality of CPUs. As such, the disclosed system may also exploit the resources of remote CPUs through a communications network or some other data communications means. In operation, the CPU fetches, decodes, and executes the instructions from the computer readable storage medium. Information, such as the computer instructions and other computer readable data, is transferred between components of the computing system via the system's main data-transfer path.

In addition, the processing system may contain a peripheral communications controller and bus, which is responsible for communicating instructions from CPU to, and/or receiving data from, peripherals as discussed herein throughout. An example of a peripheral bus is the Peripheral Component Interconnect bus that is well known in the pertinent art.

An operator display, a user interface, and a graphical user interface (GUI), as discussed throughout, may be used to display visual output and/or presentation data generated by or at the request of processing system, such as responsive to operation of the aforementioned computing programs/applications. Such visual output may include text, graphics, animated graphics, and/or video, for example. Moreover, these aspects may receive user input as discussed throughout, such as via peripherals connected to the peripheral bus.

Further, the processing system may contain a network adapter which may be used to couple to one or more communication networks, which may include or provide access to the Internet, an intranet, an extranet, or the like. Communications network may provide the processing system with means of communicating and transferring software, data and other information electronically. Network adaptor may communicate to and from the network using any available wired or wireless technologies. Such technologies may include, by way of non-limiting example, wired Ethernet or fiber optic connections, cellular WAN infrastructures such as 3G, 4G/LTE, or 5G networks, Wi-Fi, Bluetooth®, Bluetooth® Low Energy (BLE), or Zigbee® links, infrared connections, or the like.

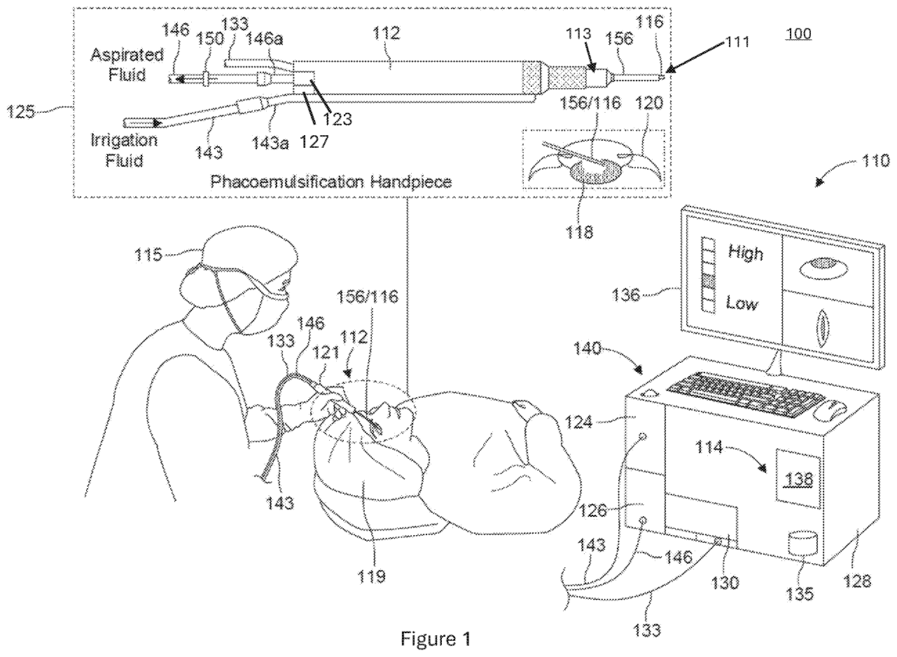

FIG. 1 is a schematic, pictorial view, along with an orthographic side view, of a system 100 having phacoemulsification system 110 (e.g., a surgical console), in accordance with an example of the present disclosure. As seen in the pictorial view of system 100, and in the schematic side view inset 125, a phacoemulsification probe 112 (e.g., a handpiece) comprises a distal end 111 (i.e., a tip) comprising a needle 116 and a coaxial irrigation sleeve 156 that at least partially surrounds needle 116 and creates a fluid pathway between the external wall of the needle and the internal wall of the irrigation sleeve, where needle 116 is hollow to provide an aspiration channel. Moreover, the irrigation sleeve may have one or more side ports at or near the distal end to allow irrigation fluid to flow toward the distal end of the handpiece through the fluid pathway and out of the port(s).

Needle 116 is configured for insertion into a lens capsule 118 of an eye 120 of a patient 119 by a physician 115 to remove a cataract. While needle 116 (and irrigation sleeve 156) are shown in inset 125 as a straight object, any suitable needle may be used with phacoemulsification probe 112, for example, a curved or bent tip needle commercially available from Johnson & Johnson Surgical Vision, Inc., Irvine, CA, USA.

Fluid, such as irrigation fluid, is controllably directed through the phacoemulsification system 110 in order to irrigate a patient's eye 120 during an ocular surgical procedure, such as a phacoemulsification procedure. During the phacoemulsification procedure, an irrigation pump 124, comprised in a console 128, pumps irrigation fluid from an irrigation reservoir (not shown) to irrigation sleeve 156 to irrigate the eye 120. The fluid is supplied in order to, for example, stabilize or maintain a certain ocular pressure in the anterior chamber of the eye 120 during surgery, as well as to provide means for fluidly transporting any particles (e.g., lens particulates that are created during emulsification) out of the eye 120. Various aspects (e.g., the flow rate, pressure, etc.) of fluid flow into and out of the eye will typically affect the surgical procedure and its outcome. The fluid is pumped via an irrigation tubing line 143 running from console 128 to an irrigation channel 143a of probe 112. In another example, pump 124 may be coupled with, or replaced by, a gravity-fed irrigation source such as a balanced salt solution bottle/bag. Fluid and waste matter (e.g., emulsified parts of the cataract) are aspirated via hollow needle 116 to a collection receptacle (not shown) by a processor-controlled aspiration pump 126, also comprised in console 128, using aspiration tubing line 146 running from aspiration channel 146a of probe 112 to console 128.

Probe 112 includes an irrigation sensor 127 (e.g., pressure sensor) coupled with irrigation channel 143a and an aspiration sensor 123 (e.g., vacuum sensor) coupled with an aspiration channel 146a. Irrigation sensor 127 may be positioned anywhere along tubing line 143 or channel 143a. Likewise, aspiration sensor 123 may be positioned anywhere along tubing line 146 or channel 146a.

Channels 143a and 146a are coupled respectively with irrigation line 143 and aspiration line 146. Pumps 124 and 126 may be any pump known in the art (e.g., peristaltic pump, progressive cavity pump). Using sensors (e.g., as indicated by sensors 127 and/or 123), a processor 138 controls a flow rate of irrigation pump 124 and/or aspiration pump 126 to maintain IOP within prespecified limits.

The phacoemulsification system 110 may include an anti-vacuum surge (AVS) module 150, coupled with the probe 112, which prevents a sudden vacuum increase being transferred to the eye in case of an occlusion break. In some cases, typically to protect against a vacuum surge hazard, the system activates AVS module 150 (seen in inset 125) to disconnect aspiration channel 146a from line 146 and aspiration pump 126. AVS module 150 can be autonomous, with a processor inside AVS 150 that receives and processes sensor readings and commands activation of module 150, or module 150 can be commanded from processor 138. To avoid vacuum surge inside the eye, the AVS module 150 very quickly (e.g., within a few tens of milliseconds) cuts off the aspiration line 146. However, blocking aspiration indefinitely by closing the valve of the AVS module 150 compromises “followability.” Followability is defined as the ability to attract cataract pieces to the phacoemulsification tip. Sufficient followability is important for efficient and safe phacoemulsification. An overly reactive system (or user) regarding vacuum surges as the tip is placed or moved within the capsular bag causes the system to fully interrupt suction (e.g., using the AVS mechanism) in cases of IOP drop, and such overreaction to AVS operation degrades followability.

To overcome or minimize the effects of a vacuum surge while maintaining acceptable followability, system 110 allows physician 115 to select a followability level. The processor 138 activates AVS module 150 with a waveform predefined according to the selected followability. In an example, user interface 140 and display 136 may be integrated into a touch screen graphical user interface (GUI). Display 136 shows a followability level scale 137 that physician 115 can use to select a given discrete level. For each user there exists some preferable followability level. Display 136 shows a discrete followability level scale 137. The physician may therefore adjust the followability level, as needed, based on experience. For example, a physician who wishes less disrupted suction power occurring by any possible false positive AVS activation events may tolerate less AVS protection, by selecting a higher followability level on the GUI. In other examples, the user may adjust the followability level with a virtual slide ruler, enter a numerical value, or make a verbal instruction. It should be appreciated that the term anti-vacuum surge (AVS) module is used herein to refer to systems for preventing a sudden outflow of fluid from the eye chamber after an occlusion break due to vacuum build up in the aspiration line caused by an occlusion at the distal end of the needle, but such systems may be referred to equivalently by other terminology such as chamber stability/stabilization system (CSS).

As noted above, processor 138 may control the flow rate of irrigation pump 124 and/or aspiration pump 126, in which one of the software modules running in processor 138 is a proportional-integral-derivative (PID) controller 114. Processor 138 estimates the IOP using readings from the irrigation pressure sensor 127 and an optional empirical offset (if the irrigation pressure is measured at the proximal end of handpiece 112). Readings of sensor 123 give the vacuum level (also called sub-pressure) and/or flow rate inside the aspiration channel. Further, the processor 138 is configured to transmit and receive control inputs from the system 110 for a surgical procedure.

Sensors 127 and 123 may be any sensor known in the art, including, but not limited to, a vacuum sensor or flow sensor. The sensor measurements (e.g., of pressure, vacuum, and/or flow) may optionally be taken close to the distal end of the handpiece where the irrigation outlet and the aspiration inlet are located, so as to provide processor 138 with an accurate indication of the actual measurements occurring within an eye, thus providing a short response time to a control loop comprised in processor 138.

In an example, the same pressure sensor model is used to measure irrigation pressure and aspiration sub-pressure, using different sensor settings/calibrations.

As further shown, phacoemulsification probe 112 includes one or more piezoelectric crystals (not shown) coupled with a horn 113, that drives needle 116 to vibrate in a resonant vibration mode that is used to break a cataract into small pieces during a phacoemulsification procedure. Console 128 comprises a piezoelectric drive module 130, coupled with the piezoelectric crystal, using electrical wiring running in cable 133. Concomitantly with the emulsification, fluid from the irrigation pump 124 is irrigated into the eye, and the irrigation fluid and emulsified crystalline lens material are aspirated from the eye by the aspiration pump 126.

Processor 138 may receive user-based commands via a user interface 140, which may include setting a vibration mode and/or frequency of the piezoelectric crystal, and setting or adjusting an irrigation and/or aspiration rate of the irrigation pump 124 and aspiration pump 126. Processor 138 may receive user-based commands via a user interface 140, which may include stroke amplitude settings for the needle 116 and commands for initiation of irrigation and/or aspiration. In an example, the physician uses a foot pedal (not shown) as a means of control. For example, a foot pedal may have a treadle that is moveable in a pitch direction and the available pitch travel may be divided into multiple functionality zones or positions. Foot pedal position one activates only irrigation, foot pedal position two activates both irrigation and aspiration, and foot pedal position three adds needle 116 vibration. Additionally, or alternatively, processor 138 may receive user-based commands from controls located in a handle 121 of probe 112.

Processor 138 may display on display 136 various measurements, criteria or settings of the system 110, such as the type of procedure, the phase of the procedure and duration of the phase, various parameters such as vacuum, flow rate, power, and values that may be input by the user, such as tube length (irrigation and aspiration), tip size, and vacuum rate. The GUI of the user interface 140 and display 136 may allow a user to monitor the characteristics of the system 110 and/or control or select settings or criteria for various components of the system 110.

Some or all of the functions of processor 138 may be combined in a single physical component or, alternatively, implemented using multiple physical components. These physical components may comprise hard-wired or programmable devices, or a combination of the two. In some examples, at least some of the functions of processor 138 may be carried out by suitable software stored in a memory 135 (as shown in FIG. 1). This software may be downloaded to a device in electronic form, over a network, for example. Alternatively, or additionally, the software may be stored in tangible, non-transitory computer-readable storage media, such as optical, magnetic, or electronic memory.

The apparatus shown in FIG. 1 is simplified for clarity of presentation. For example, the disclosed AVS scheme may be applied using alternative or additional control devices of the system, such as other valves (e.g., a bypass valve). Examples of the present invention, however, are by no means limited to this specific sort of example phacoemulsification system, and the principles described herein may similarly be applied to other sorts of phacoemulsification or other suitable types of surgical systems.

In examples, the GUI of display 136 provides feedback to the user should the pre-determined or automatic settings, variables, or criteria need adjustment to ensure all the desired settings of the system. The GUI, such as the GUI of display 136, may then permit the user to change or modify those settings accordingly. Parameters/settings may include at least aspiration, vacuum, and ultrasound power, wherein ultrasound power controls the handpiece tip oscillation. Many surgeons exercise their skills, techniques, and experiences to navigate through surgery by manually modifying parameters, as there may not be a fixed procedure to follow. Alternatively, surgeons may follow a partially or fully automated control of parameters during surgery. However, in this latter case, surgery, and thus the setting of and monitoring of surgical parameters, may still vary from patient to patient.

Thus, whether a surgery is automatically or manually controlled, various issues may occur during surgery. For example, high ultrasonic energy for a particular patient may cause damage to the corneal endothelial cells and lead to corneal decompensation. Excessive heat build-up from too much ultrasonic power into the eye or improper aspiration flow during surgery may lead to corneal burns or damage other delicate ocular structures.

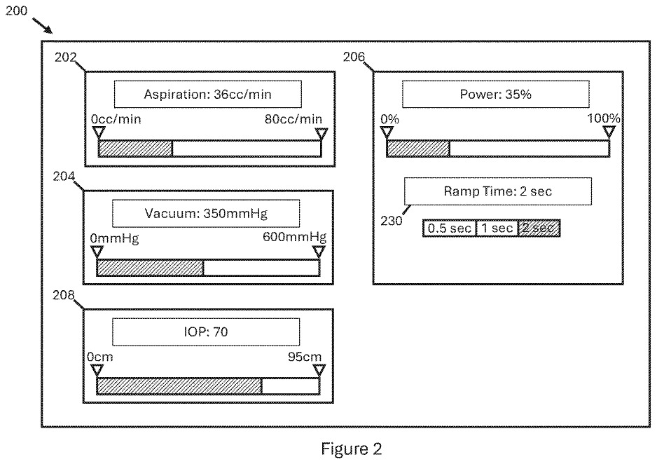

FIG. 2 shows an example graphical user interface (GUI) 200 for automated phacoemulsification. The GUI 200 may be displayed on display 136 of system 110. During phacoemulsification, the GUI 200 may display various parameters, including aspiration levels 202, vacuum levels 204, power levels 206, and intraoperative pressure (IOP) 208. Phacoemulsification parameters may be entered or selected by the user via the GUI 200.

The user may be presented with suggested prefilled parameters. Alternatively, the user may be presented with parameters previously saved by the user. In one example, the parameter may include, but is not limited to, max power setpoint, max aspiration setpoint, max vacuum setpoint, vacuum threshold, and/or power ramp time. The parameters selected via the GUI 200 and stored by the system 110 on which resides at least a portion of processor 138 are applied automatically when the phacoemulsification algorithm is activated during the procedure or surgery.

By way of example, the max power setpoint may be the maximum power percentage (from 0-100%) output through the handpiece when operating in ultrasound mode. The max aspiration setpoint may be the maximum aspiration flow, e.g., from 0-80 cc/minute output through the handpiece. The max vacuum setpoint may be the maximum available vacuum, from e.g., 0-600 mmHg, to pull and remove lens particles from the eye. For example, the vacuum threshold may be the threshold that dictates the flow rate and/or vacuum level that signifies a “full occlusion,” which signals the system to apply more power starting from a set percentage of the max power setpoint to the max power setpoint. Power ramp time 230 may specify how fast the power will ramp up (e.g., from 35% of the max power setpoint to the max power setpoint). By way of non-limiting example, the power ramp time 230 may have three options, such as 0.5 seconds, 1 second, and 2 seconds, to ramp up the power.

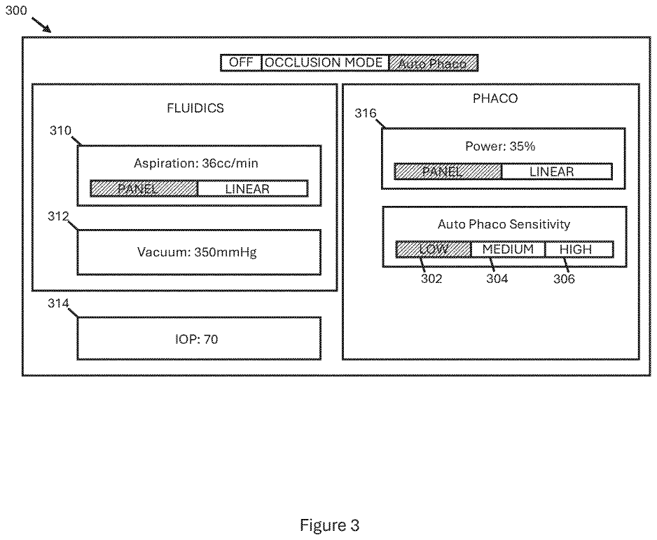

FIG. 3 shows an example GUI 300 for phacoemulsification that includes automated phacoemulsification sensitivity settings. The GUI 300 may be displayed on display 136 of system 110. As shown in FIG. 3, the user can select low sensitivity 302, medium sensitivity 304, or high sensitivity 306. Further and by way of example, the user may adjust settings related to fluidics 310, vacuum 312, IOP 314, and/or power 316. Of additional note, the phacoemulsification system may provide an anti-vacuum surge (AVS) module (also known as a chamber stability system (CSS)), such as the AVS devices and modules described in U.S. Pat. No. 11,771,818 and U.S. application Ser. No. 17/511,166, which are incorporated herein by reference in their entirety.

By way of example, in manual phacoemulsification, it may be necessary to “feather” the amount of power provided to the lens using a foot pedal to break-up or emulsify and remove the lens, which vary in size and density. In automated phacoemulsification, feathering may not be needed because an algorithm may start and apply power and aspiration based on the measured flow rate and/or vacuum level relative to the algorithm-defined vacuum thresholds. Thus, as the surgeon works to bring a particle to the tip, the automated phacoemulsification system may compare vacuum levels and determine when and how to apply power. That is, in automated phacoemulsification mode, the aspiration readings may be compared by the processor 138 of the system 110 to the vacuum threshold level(s), and the power level may be adjusted accordingly.

Automated systems, such as those referenced above, are designed to help eliminate or reduce the aforementioned issue of damage to the corneal endothelial cells, leading to corneal decompensation. Further, as referenced above, when the electrical energy at the surgical tip is converted to mechanical energy for emulsification, it is also converted to heat, and excessive heat build-up from too much power being applied in the eye may lead to corneal burns and/or heat damage to delicate ocular structures.

For example, surgical wound burns may compromise corneal integrity, contribute to astigmatism, and produce scarring. Other than fully automating the surgery, current mitigation efforts include surgical techniques which reduce friction at the sides of the surgical wound, generally decrease ultrasonic power, or increase flow rates using the available fluidic controls discussed above. However, these are largely subjective mitigations and thus cannot typically be translated to scale to address wound burn risk. Objective mitigations might include, by way of non-limiting example, controlling tip temperature, controlling thermal load, and the like.

In an example of prior mitigation methods, U.S. Pat. No. 8,308,676 details the use of temperature sensors to sense the aspiration fluid, and to calculate therefrom an energy balance. In this energy balance, energy enters the eye via the ultrasound power, and is removed from the eye via fluid.

In other methods, flow and power measurements sensing are used to determine whether current operating conditions fit within allowable power levels and duty cycles. This fit is used to modify a handpiece's operation to modify the fluidic flow rates, and to alert a surgeon when conditions are beyond proper limits.

The disclosed system and method of mitigating eye damage from heating of the surgical tip obviates the need for sensors by using a temperature estimation algorithm for the phaco-tip, with the mathematical model of the algorithm informed by real-time surgical parameters. Moreover, the disclosed solution continuously approximates equilibrium states, thereby understanding energy delivery and flow rates simultaneously to appreciate the impact thereof on the surgical site temperature.

The disclosed system and method may, in addition to algorithmic assessment of temperatures, involve sensing, control, or warning/alert features to further mitigate the potential of corneoscleral wound burns. Further, the disclosed solution may enable a surgical team to customize various settings, such as to account for a given set of ambient room conditions, patient sensitivity, or cataract severity, by way of non-limiting example.

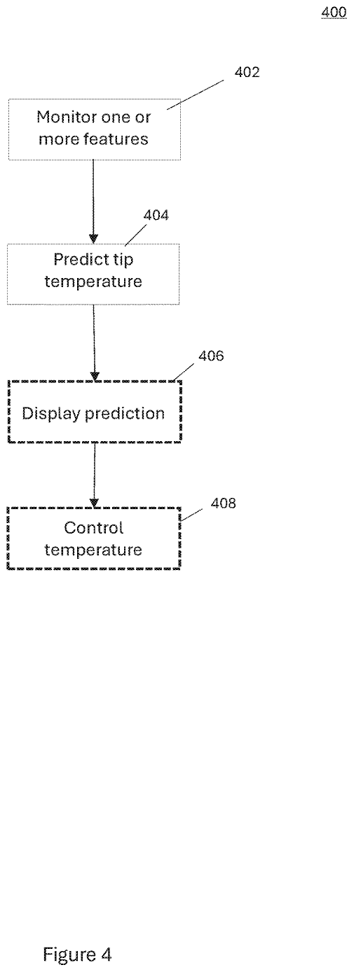

More particularly, the examples may calculate a continuous estimation of the phaco-tip temperature through a method 400, as illustrated in FIG. 4. In one or more examples, one or more processors, such as processor 138 of system 110, may utilize one or more modeling algorithms to implement the method 400. For example, one or more processors monitor one or more features at step 402 of the system 110, such as, but not limited to current phaco-power levels/settings, fluidics (i.e., aspiration/irrigation) settings, characteristics of emulsification, and the like. Emulsification characteristics of emulsified particles may include, for example, but not limited to, density, size, temperature, number of particles, and the like.

The modeling algorithm(s) make tip temperature predictions at step 404. The tip temperature predictions may refer to current or estimated temperatures of the tip of a handpiece, such as the needle 116 of the phacoemulsification probe 112. The tip temperature predictions may be based on one or more of the monitored phaco-power level/settings, fluidics settings (irrigation/aspiration flow rate, pressure, vacuum, etc.), emulsification characteristics, and the like. Additionally or alternatively, the tip temperature predictions may be based on one or more prior tip temperature predictions and/or mitigations steps performed on the system 110 during previous procedures. In some examples, the modeling algorithm(s) may provide the tip temperature prediction within calculation limits set by the modeling algorithm(s) and/or a health care provider, such as a surgeon. Calculation limits may, for example, be based on information, such as, but not limited ambient room conditions, patient sensitivity, cataract severity, and the like, gathered during a sampling window of time.

The predicted tip temperatures may be displayed, such as graphically, on a GUI, at step 406. In some examples, the predicted tip temperatures may be displayed on the GUI as part of a heat gauge, a heat map versus time, or another graphical representation of the tip in real-time during surgery. Further, the predicted tip temperature may be displayed on the GUI as part of a graphical representation of trending temperatures of the tip during surgery. It is noted that the tip temperature predictions may be displayed in a variety of formats, such as, but not limited to, numerically, being color coded, and the like.

In one or more examples, temperature of the tip may be controlled by controlling the phaco mode or power delivery to the handpiece tip at step 408. The temperature of the handpiece tip may be controlled based at least in part on the predicted tip temperatures. In some examples, one or more features (e.g., phaco-power level/settings, fluidics settings, and the like) to control the phaco mode may be selectable via the GUI. The phaco mode may continuously and/or automatically modify the phaco power delivery to optimize surgery. For example, when the phaco mode is selected to control temperature of the tip, the phaco power delivery may be automatically decreased as necessary when the modeling algorithm(s) estimate unacceptable temperature increases (e.g., based on predicted tip temperatures and/or real-time temperatures of the tip). The sensitivity level for such automated mitigations may be set by the surgeon, such as depending on the user's preferences or surgical requirements. For example, only when the aspiration flow rate is below 10 cc/min for at least 3 seconds may the system begin to reduce power by certain pre-determined amount if the tip temperature is not within a reasonable range. Controlling the phaco mode allows the surgeon to optimize phaco power delivery to the wound with respect to the temperature while efficiently removing cataract particles from the anterior chamber. It is noted that controlling the phaco mode is described as being selectable; however, it should be understood that examples are contemplated herein in which phaco mode is automatically controlled without receiving a user's selection.

In one or more examples, the GUI may display temperature metric(s), allowing the surgeon to map out tip temperature(s) for phaco submodes. For instance, the tip temperature metrics may be used to assess power level and flow conditions that lead to a particular temperature range or ranges during different aspects of the surgery. The tip temperature metric(s) may vary based on the power setpoint, the incision size and/or type, chamber volume, and wound leakage, and/or other surgical variables. Further, providing the tip temperature metric(s) on the GUI allows the surgeon to refine technique over time for certain conditions, which leads to better post-operative outcomes, such as improved corneal clarity.

Output from the modeling algorithm(s) and/or indicated via the GUI may be used to mitigate and control temperature changes. For example, based on the output, mitigations may be performed, such as in the form of alerts to forewarn a surgeon should the modeling algorithm(s) determine or predict that the estimated temperature is rising, or will rise, to within a range that may create a potential for wound burn. The alert may be, for example, graphical, audio, instructional, or the like. The parameters of the alert may be configurable by a user, and as such may be uniquely a part of a surgeon's preferred profile, and/or may be enabled or disabled for the standard or auto phaco modes discussed above, by way of non-limiting example.

Additionally or alternatively, based on the output, mitigations for an unsafe condition may include, by way of example, a modification to one or both of ultrasound/phaco power and fluidics to control temperature changes. The modifications may be performed either automatically or as may be manually indicated by the surgeon, such as upon the occurrence of an alert. However, ultrasound power delivery may not be stopped completely unless specifically set to do so by surgeon/staff when the tip temperature is within a certain range, is projected to reach a certain range, or reaches a predetermined threshold during surgery. Further, such as for the auto phaco mode, the system 110 may go into a thermal-safe mode for mitigation and may accordingly suspend all phaco power delivery for a certain period of time, or may halt power delivery entirely/for a longer period if a higher tip temperature persists, such as due to subsystem malfunction or the like.

It is noted that embodiments are contemplated herein in which a tip temperature is predicted and the temperature of the tip is controlled, as described herein, without displaying the predicted tip temperature on a GUI. That is, once the system 110 predicts the tip temperature, the system 110 may control the phaco mode or power delivery to the handpiece tip to control the tip temperature, bypassing the display of the tip temperature predictions. Further, it is noted that embodiments are contemplated in which the system 110 predicts the tip temperature and displays the prediction on the GUI. As such, a user may manually control one or more features of the phaco mode, power delivery, and/or fluidic settings to control the tip temperature. Accordingly, FIG. 4 illustrates steps 406 and 408 in dashed lines as being optional.

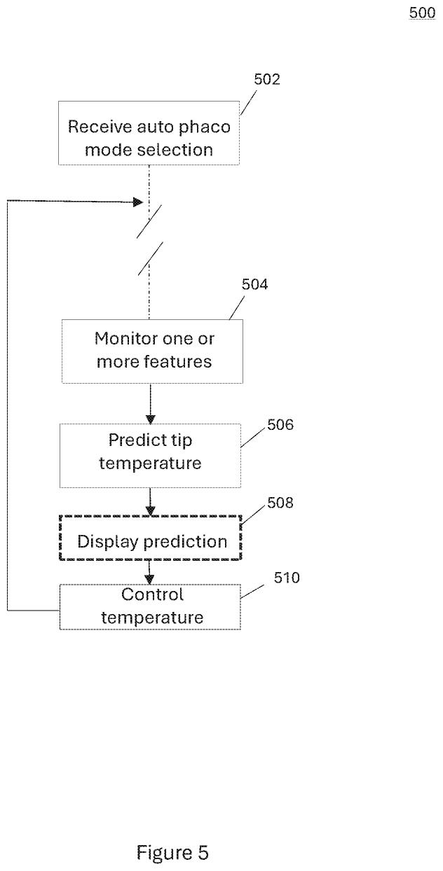

In the case of thermal monitoring during auto phaco mode 500, and as illustrated in the flow diagram of FIG. 5, the surgeon may select auto phaco mode, in which the system 110 receives the selection and places the system 110 into the auto phaco mode at step 502. In auto phaco mode, the phaco power is automatically adjusted in proportion to the aspiration as discussed herein. Accordingly, in auto phaco mode the surgeon relies on the system 110 to control output power, rather than feathering the controls (e.g., foot pedal) as may be done in non-automated surgeries.

The cataract being removed may be more or less dense. A denser cataract requires more significant use of phaco power to break up the lens material. The tip occlusion may be longer in the case of a higher density cataract, or from the presence at the tip of a denser cataract fragment, or if a clog has formed near the tip or at the aspiration fitting resulting in minimal or no aspiration flow. In such cases, more heat builds up at the wound site.

Of note, the method of FIG. 5 may operate in a manner similar to the method of FIG. 4. Further, the system 110 may continuously estimate the tip temperature at the point of intersection with the surgical wound, and may continuously apply this information to known acceptable ranges for phaco power in light of one or more monitored features and/or output from the modeling algorithm(s) and/or indicated via the GUI. In light of this data, the system 110 mitigates and controls temperature changes at step 510. For instance, controlling the tip temperature may be performed, such as in the form of providing alerts and/or performing modifications to ultrasound/phaco power and fluidics settings. The mitigations may also be based on whether the surgery is performed in standard or auto phaco mode. The mitigations may include, by way of non-limiting example: a warning to the user that the surgical wound may be overheating; a reduction of ultrasonic power as a function of the projected temperature increase; progressing the system into a “safe state”; and optionally preventing additional phaco power delivery until the safe state has been reached and then exited, as discussed herein in relation to FIG. 4. Additionally, similar to that of FIG. 4, embodiments are contemplated herein in which a tip temperature is predicted (e.g., at step 506) and the temperature of the tip is controlled (e.g., at step 510) without displaying the predicted tip temperature on a GUI. Accordingly, FIG. 5 illustrates step 508 in dashed lines as being optional.

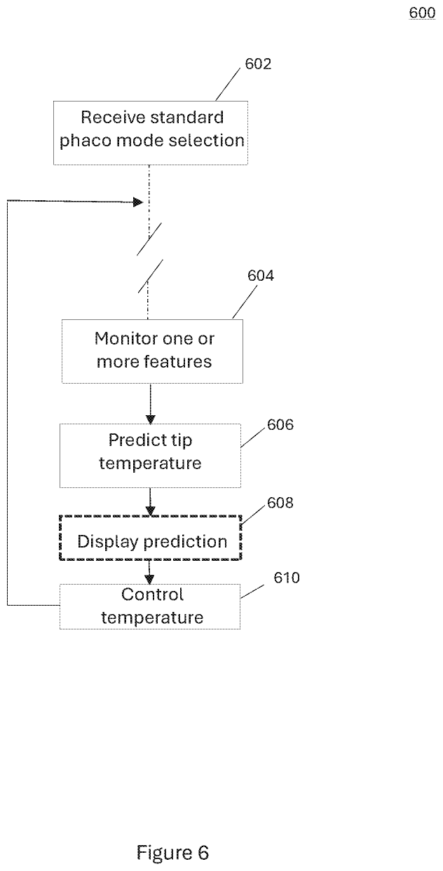

Similarly, thermal monitoring may occur in standard mode 600, as shown in FIG. 6. In this flow, the user may select standard phaco mode selection, in which the system 110 receives the selection and places the system 110 in standard, manual phaco mode at step 602. As was described herein, a harder cataract, clog, or reduction in aspiration flow rate requires the use of more phaco power to fully emulsify the cataractous material. These issues may cause the surgical wound to heat due to the lack of cooling provided by the flow of fluid in and/or out of the eye.

When this heating occurs, the system 110 continuously, and/or in a sampling timeframe indicated as a calculation limit, estimates/predicts the tip temperature in light of various factors discussed throughout, and may assess that there has been an excessive temperature increase. Such features that predict, display, and control temperatures as described with respect to FIG. 6 operate in a manner similar to the method of FIG. 4. The system in manual mode may further perform the mitigation and temperature control techniques described throughout, and/or may request action manually for some mitigations, and/or, particularly in more severe circumstances, may override and automate certain mitigations. Mitigation and temperature control techniques may include user alerts, phaco-power modifications, aspiration modifications, or safe mode, by way of non-limiting example.

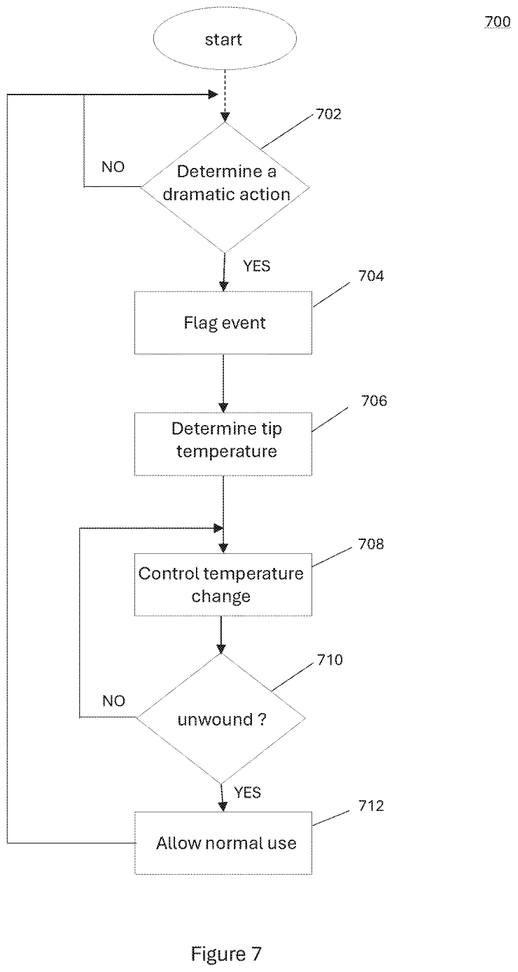

FIG. 7 is a flow diagram 700 illustrating additional actions for thermal monitoring after particular surgical actions. Initially, the system, such as system 110, may determine whether a dramatic action occurred at step 702. By way of example, this dramatic action may be a significant and/or uncommon surgical event, such as the surgeon removing the handpiece away from the patient, thus exiting the surgical wound. This may be done, for example, in exceptional cases so as to prevent risk of injuries. Upon detection of this dramatic change in the surgical parameters, such as a dramatic change in the intraoperative pressure, the system may flag this event at step 704.

Following this dramatic action, the system may determine tip temperatures at 706. To determine tip temperatures, the system may operate in a same or similar manner to the method of FIG. 4. For instance, the system may monitor one or more features, predict tip temperatures, display predictions, and control temperatures, as described with respect to method 400. In one or more examples, the system may estimate/predict real-time tip temperatures based on a secondary (i.e., different) model in view of the substantially different circumstances for the phaco tip, such as a heat convection model. Temperature change is controlled at step 708. Temperature change may be mitigated and controlled in a same or similar manner as the mitigations described herein. Once the temperature is determined to have decreased to a safe level and the mitigations (i.e., alert, phaco power modifications, or safe mode) unwind at step 710, the system allows the surgeon to resume normal use of the system at step 712 to continue, for example, the phacoemulsification procedure.

Of course, in the foregoing examples, rather than triggering on modeled estimates, the warning, phaco control, safe mode activation, or other mitigations may also be triggered with an actual tip temperature measurement. This may be achieved by means of a thermocouple, thermistor, or some other temperature monitoring technology at the tip of the instrument or in the consumable, by way of non-limiting example.

Alternative features may include the integration of environmental characteristics of the surgery, and the inclusion thereof of those characteristics in the modelling discussed throughout. By way of example, ambient temperature measurements, as well as fluid temperatures, may affect the modelling. Further, the systems and methods discussed throughout may be employed in other fields and with other surgical equipment, such as use in ultrasonic dental handpieces that may also implement irrigation and/or aspiration.

Simply put, the algorithmic modelling provided herein gauges how much energy has been delivered to, and removed from, the tip over time, to enable the calculation of tip temperature over time. Relatedly, this tip temperature corresponds to a likelihood of damage to the eye or the surgical wound over time. Thereby, the estimation of the temperature allows for an avoidance of damage as a result of surgery.

As mentioned throughout, this modelling thus allows for mitigating action, either by alerting the user of the surgical console of a current or upcoming likelihood of damage and recommending mitigating/corrective action, and/or by alerting the user, and/or by automating corrective action. Thus, the disclosure is directed to both auto phaco and standard operating modes, in the exemplary use-case of a phacoemulsification surgery.

More specifically, the examples may use an algorithm based on a lumped element, or lumped capacitance, model. This model assumes that heat transfer occurs at the surface of the phaco tip, i.e., at the junction point thereof with the surgical wound. This model assumes that heat resistance is small, given the exceedingly small distance between the heated point and the surface of interest, i.e., the surgical wound. With these assumptions, the model assumed is:

$$T ( t ) = {T}_{env} + ( T ( 0 ) - {T}_{env} ) {e}^{- rt}$$

In this equation, the “r” and the delta T (i.e., T(0)−Tₑₙᵥ) may be empirically derived. Further, it should be noted, as referenced above, that other factors may affect the heat transfer model. Such factors may include, by way of non-limiting example, cooling from the ambient air, cooling from the irrigation, and heat provided by non-surgically related energy modes of the phaco/ultrasound device.

Needless to say, experiential data may provide feedback to modify the model above, or any model employed, to better estimate tip temperature. As is evident from the discussion herein, the model employed by the processor 138 and corresponding memory 135 of system 110 may be a closed loop system. As such, feedback may occur at each console at which surgery is performed, and for each surgery performed at each console. Accordingly, the model may additionally have a learning module associated therewith, such as an artificial intelligence (AI) module.

More specifically, the AI module may monitor for eye damage or tip temperature spikes, such as may be indicated by sensors or surgeon input, that occur when the model says they should not, and/or lack of eye damage or lack of tip temperature rise that is contrary to the model. In such cases, the AI module may adjust the model accordingly. This adjustment may occur in the self-contained environment of a single console, or based on variations from the model assessed across multiple consoles, such as within a care facility, within a health-care network, or globally for example

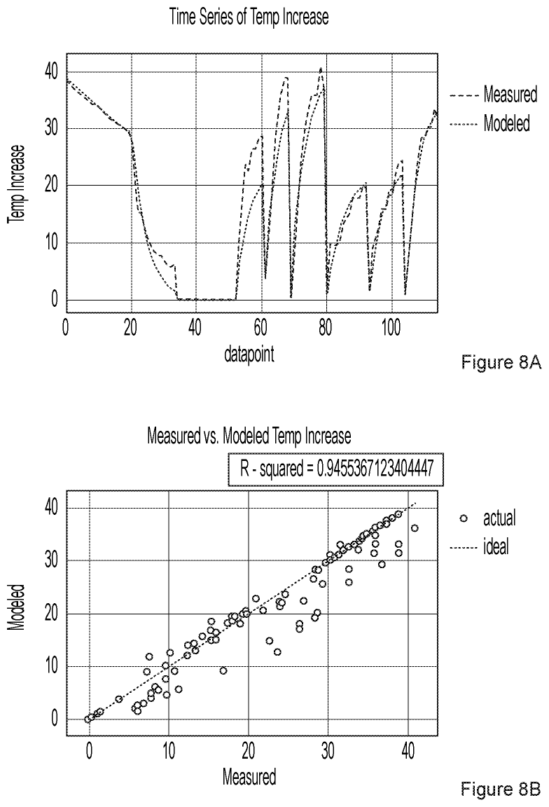

FIGS. 8A and 8B graphically illustrate this model's performance versus the actual temperature measurement at the phaco tip. As shown, the fit of the model to the actual temperature is highly correlated. This is the case for all conditions, including a hot tip with ambient cooling, a hot tip with aspirated cooling, and a room temperature tip during phacoemulsification, both with and without aspiration.

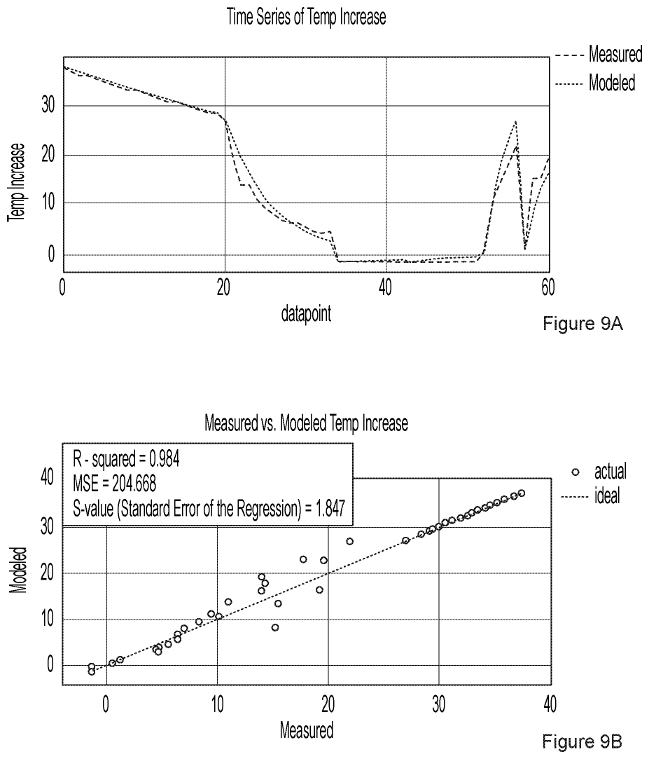

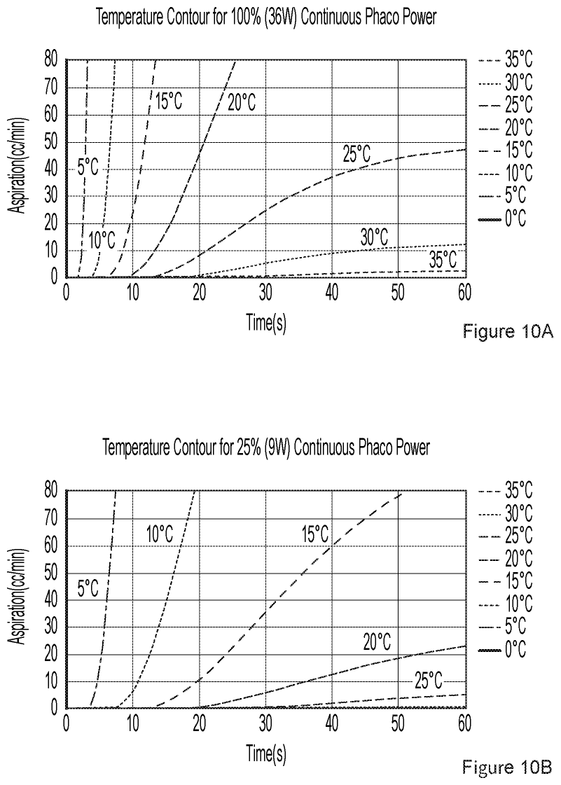

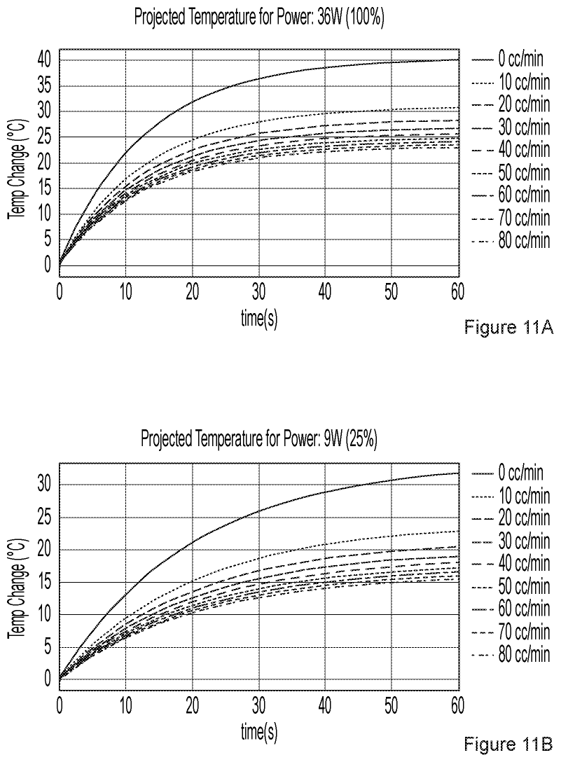

Similarly, FIGS. 9A and 9B illustrate the particular fit of this model at 50% phaco power (e.g., WHITESTAR™ Power) and at 0-15 cc/min of aspiration. FIGS. 10A and 10B illustrate the temperature contour curves for time versus aspiration at 100% and 25% continuous phaco power, respectively. FIGS. 11A and 11B illustrate the aspiration contour curves for time versus temperature change, also at 100% and 25% power, respectively.

Simply put, the model may perform its estimation using, at least in part, a successive approximations approach to the formula set forth above. That is, the next point estimate on the temperature curve, y(t+Δt), is estimated as the prior point, y(t), plus an estimated increment based on phaco power level and aspiration flow rate.

In the simplest case, for known values of Δt in an exponential function, the relationship of y(t+Δt) and (t) is linear and does not depend strictly on (t). For example, in the simple case of a decaying exponential e\hat{ }−t, the relationship between the prior point and the next point falls on a line y(t+Δt)=(e\hat{ }(−Δt))(y(t)), independent of (t).

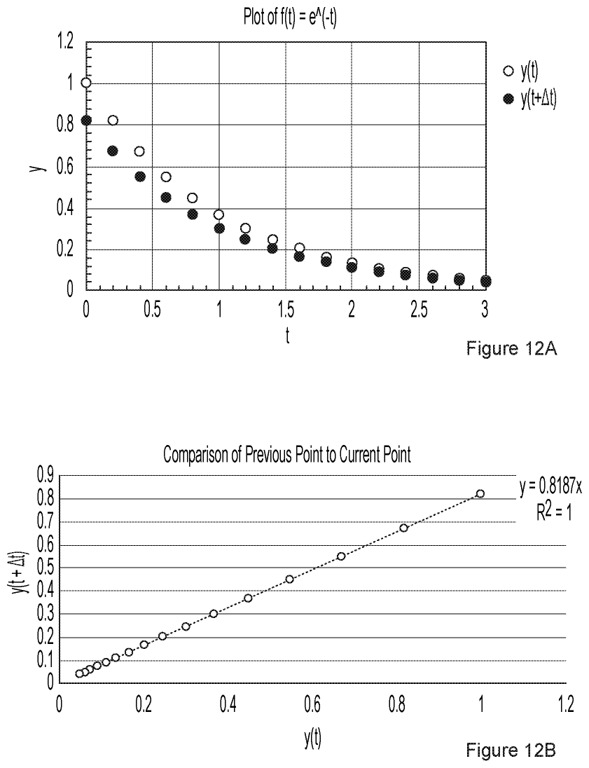

Table 1, below, represents the outcome of the above equation for a variety of values of Δt. By way of example, for Δt, t is as represented in the table, and y_next=0.81873*y_previous according to the equation above. Application of this constant to assess the next estimated point based on the prior estimated point is further illustrated in FIGS. 12A and 12B.

TABLE 1

| t | y(t) | y(t + Δt, calculated) | e\hat{ }(−Δt) |
|---|---|---|---|
| 0 | 1 | 0.818730753 | 0.818730753 |
| 0.2 | 0.81873 | 0.670320046 | 0.818730753 |
| 0.4 | 0.67032 | 0.548811636 | 0.818730753 |
| 0.6 | 0.54881 | 0.449328964 | 0.818730753 |
| 0.8 | 0.44933 | 0.367879441 | 0.818730753 |
| 1 | 0.36788 | 0.301194212 | 0.818730753 |
| 1.2 | 0.30119 | 0.246596964 | 0.818730753 |

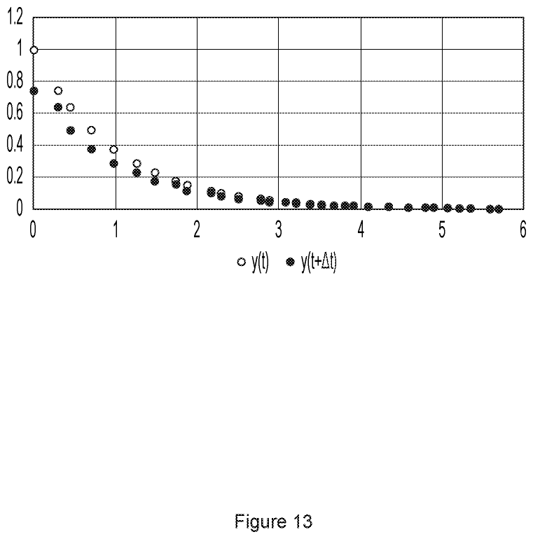

In a more complex case, the sampling times may vary. In such a case, the equation y(t+Δt)=(e\hat{ }(−Δt))(y(t)) is still obeyed, but (e\hat{ }(−Δt)) must be recalculated by the model for each successive approximation, as the multiplier for the next point from the prior point will not be a constant for variable Δt. This is illustrated in Table 2, and with reference to FIG. 13.

TABLE 2

| t | y(t) | y(t + Δt, calculated) | e\hat{ }(−Δt) |
|---|---|---|---|
| 0 | 1 | 0.823061043 | 0.823061043 |
| 0.19472 | 0.82306 | 0.618948778 | 0.752008351 |
| 0.47973 | 0.61895 | 0.477543995 | 0.771540412 |
| 0.7391 | 0.47754 | 0.405601388 | 0.849348735 |
| 0.90238 | 0.4056 | 0.322351843 | 0.794750345 |
| 1.13211 | 0.32235 | 0.24438755 | 0.758139143 |
| 1.409 | 0.24439 | 0.199864303 | 0.81781704 |

### EXAMPLES

### Example 1

The disclosure includes a phacoemulsification system (100), comprising: a surgical handpiece (112) configured to emulsify lens material in an eye; an aspiration line (146) coupled with the handpiece; and a surgical console (110) communicatively coupled with the aspiration line and the surgical handpiece, the surgical console configured to estimate temperature of a tip (111) of the surgical handpiece, and to modify at least one of ultrasound power to the surgical handpiece, a flow rate of the aspiration line, or a vacuum level of the aspiration line responsively to the estimated temperature meeting a predetermined temperature threshold.

### Example 2

The disclosure includes a phacoemulsification system (100), comprising: a surgical console (110) including a graphical user interface (GUI) (136, 140) and at least one processor (138) configured to transmit and receive control inputs for a surgical procedure; and a surgical handpiece (112) having a plurality of fluidic (143, 146) and electrical lines (133) communicative with the surgical console (110). The surgical handpiece (112) comprises: a surgical tip (111) comprising a needle (116) and a sleeve (156), wherein the surgical tip is configured for emulsification of lens material in an eye; an ultrasonic horn (113) configured to drive the surgical tip (111) via the electrical line responsive to a first of the control inputs; one of the plurality of fluidic lines being an aspiration line (146) from the tip substantially along a length of the surgical handpiece, the aspiration line configured for aspiration of the lens material from the eye responsive to a second of the control inputs; a second of the plurality of fluidic lines being an irrigation line (143) from proximate to the tip substantially along a length of the surgical handpiece; the irrigation line configured for irrigation of the eye responsive to a third of the control inputs. The at least one processor (138) is configured to read non-transitory computing code which, when executed by the at least one processor of the surgical console, causes the steps of: monitoring power applied to the ultrasonic horn; monitoring the aspiration; monitoring the irrigation; applying a model estimating a current and an upcoming temperature of the surgical tip based collectively on the monitoring steps; and providing mitigation if the current or the upcoming temperature is estimated by the model to be higher than a predetermined threshold likely to cause injury.

### Example 3

The disclosure includes a computer-based surgical support method (500, 600, 700, 800) comprising executing computing code associated with a surgical console (110), which executing performs the steps of: receiving aspiration data regarding aspiration; receiving characteristics of a surgical procedure, including at least a power level of a surgical instrument (112) associated with the surgical procedure; applying a model estimating temperature of the surgical instrument based collectively on the receiving steps; and mitigating adverse patient effects if the temperature is estimated by the model as likely to cause injury.

### Example 4

The disclosure includes the system and method according to any one of Examples 1-3, wherein the temperature estimate is further responsive to characteristics of the eye lens being emulsified.

### Example 5

The disclosure includes the system and method according to any of Examples 1-4, wherein the surgical console applies a model obeying the equation: T(t)=Tₑₙᵥ+(T(0)−Tₑₙᵥ) e^{-rt}.

### Example 6

The disclosure includes the system and method according to any of Examples 1-5, wherein the surgical console (110) is further configured to alert to a surgeon at the predetermined patient injury threshold.

### Example 7

The disclosure includes the system and method according to any of Examples 1 and 3-6, an irrigation line (143) coupled to the handpiece (112), wherein the surgical console is further configured to modify an irrigation level of the irrigation line responsive to the meeting of the temperature threshold.

### Example 8

The disclosure includes the system and method according to any of Examples 1-7, wherein the predetermined patient injury threshold is at a temperature indicative of surgical wound burns.

### Example 9

The disclosure includes the system and method according to any of Examples 1 and 3-8, wherein the tip (111) of the surgical handpiece (112) comprises a needle (116) and a sleeve (156), the tip being configured for emulsification of the lens material, and wherein the system further comprises an ultrasonic horn (113) configured to drive the tip of the handpiece based on a modification of the ultrasound power provided to the surgical handpiece.

### Example 10

The disclosure includes the system and method according to any of Examples 1 and 3-9, wherein the surgical console (110) comprises at least one processor and a non-transitory computer-readable storage medium comprising one or more modeling algorithms that when executed by the at least one processor, cause the at least one processor to: apply the one or more modeling algorithms to estimate a current and an upcoming temperature of the tip based on at least one of the flow rate or the vacuum level of the aspiration line; and provide mitigation if the current or the upcoming temperature is estimated by the one or more modeling algorithms to be higher than the predetermined threshold likely to cause injury.

### Example 11

The disclosure includes the system and method according to any of Examples 1-10, wherein monitoring the aspiration comprises monitoring one or more of the flow rate or the vacuum level of the aspiration line (146), and wherein monitoring the irrigation comprises monitoring a flow rate of the irrigation line (143).

### Example 12

The disclosure includes the system and method according to any of Examples 1-11, wherein at least one processor (138) is further configured to read the non-transitory computing code to cause the step of monitoring characteristics of the emulsification, including the characteristics of the particles.

### Example 13

The disclosure includes the system and method according to any of Examples 1-12, wherein the characteristics of the emulsification comprise a density of the lens material.

### Example 14

The disclosure includes the system and method according to any of Examples 1-13, wherein the model comprises a lumped element model.

### Example 15

The disclosure includes the system and method according to any of Examples 1-14, wherein the mitigation comprises an alert to a surgeon.

### Example 16

The disclosure includes the system and method according to any of Examples 1-15, wherein the mitigation comprises at least one of modification to the power, a level of the aspiration, and a level of the irrigation.

### Example 17

The disclosure includes the system and method according to any of Examples 1-16, wherein the injury is a surgical wound burn.

Although the examples described herein mainly address phacoemulsification procedures, the methods and systems described herein can also be used in other medical applications. Further, the various examples described above are provided by way of illustration only, and should not be construed to limit the claims attached hereto, including to any specific type of surgical procedure.

Further, the descriptions of the disclosure are provided to enable any person skilled in the art to make or use the disclosed examples. Various modifications to the disclosure will be readily apparent to those skilled in the art based on this disclosure, and the generic principles defined herein may be applied to other variations without departing from the spirit or scope of the disclosure. Thus, the disclosure is not intended to be limited to the examples and designs described herein, but rather is to be accorded the widest scope consistent with the principles and novel features claimed as follows.

What is claimed is:

1. A phacoemulsification system, comprising:
a surgical handpiece configured to emulsify lens material in an eye;
an aspiration line coupled with the handpiece; and
a surgical console communicatively coupled with the aspiration line and the surgical handpiece, the surgical console configured to estimate temperature of a tip of the surgical handpiece, and to modify at least one of ultrasound power to the surgical handpiece, a flow rate of the aspiration line, or a vacuum level of the aspiration line responsively to the estimated temperature meeting a predetermined temperature threshold.

2. The system of claim 1, wherein the temperature estimate is further responsive to characteristics of the eye lens being emulsified.

3. The system of claim 1, wherein the surgical console applies a model obeying the equation:
$$T ( t ) = {T}_{env} + ( T ( 0 ) - {T}_{env} ) {e}^{- rt} .$$

4. The system of claim 1, wherein the surgical console is further configured to alert to a surgeon at the predetermined patient injury threshold.

5. The system of claim 1, further comprising an irrigation line coupled to the handpiece, wherein the surgical console is further configured to modify an irrigation level of the irrigation line responsive to the meeting of the temperature threshold.

6. The system of claim 1, wherein the predetermined patient injury threshold is at a temperature indicative of surgical wound burns.

7. The system of claim 1, wherein the tip of the surgical handpiece comprises a needle and a sleeve, the tip being configured for emulsification of the lens material, and
wherein the system further comprises an ultrasonic horn configured to drive the tip of the handpiece based on a modification of the ultrasound power provided to the surgical handpiece.

8. The system of claim 1, wherein the surgical console comprises at least one processor and a non-transitory computer-readable storage medium comprising one or more modeling algorithms that when executed by the at least one processor, cause the at least one processor to:
apply the one or more modeling algorithms to estimate a current and an upcoming temperature of the tip based on at least one of the flow rate or the vacuum level of the aspiration line; and
provide mitigation if the current or the upcoming temperature is estimated by the one or more modeling algorithms to be higher than the predetermined threshold likely to cause injury.

9. A phacoemulsification system, comprising:
a surgical console including a graphical user interface (GUI) and at least one processor configured to transmit and receive control inputs for a surgical procedure;
a surgical handpiece having a plurality of fluidic and electrical lines communicative with the surgical console, the surgical handpiece comprising:
a surgical tip comprising a needle and a sleeve, wherein the surgical tip is configured for emulsification of lens material in an eye;
an ultrasonic horn configured to drive the surgical tip via the electrical line responsive to a first of the control inputs;
one of the plurality of fluidic lines being an aspiration line from the surgical tip substantially along a length of the surgical handpiece, the aspiration line configured for aspiration of the emulsified lens material from the eye responsive to a second of the control inputs; and
a second of the plurality of fluidic lines being an irrigation line from proximate to the surgical tip substantially along a length of the surgical handpiece, the irrigation line configured for irrigation of the eye responsive to a third of the control inputs;
wherein the at least one processor is configured to read non-transitory computing code which, when executed by the at least one processor of the surgical console, causes the steps of:
monitoring power applied to the ultrasonic horn;
monitoring the aspiration;
monitoring the irrigation;
applying a model estimating a current and an upcoming temperature of the surgical tip based collectively on the monitoring steps; and
providing mitigation if the current or the upcoming temperature is estimated by the model to be higher than a predetermined threshold likely to cause injury.

10. The system of claim 9, wherein monitoring the aspiration comprises monitoring one or more of a flow rate or a vacuum level of the aspiration line, and wherein monitoring the irrigation comprises monitoring a flow rate of the irrigation line.

11. The system of claim 9, wherein the at least one processor is further configured to read the non-transitory computing code to cause the step of monitoring characteristics of the emulsification, including the characteristics of the particles.

12. The system of claim 11, wherein the characteristics of the emulsification comprise a density of the lens material.

13. The system of claim 9, wherein the model comprises a lumped element model.

14. The system of claim 13, wherein the lumped element model obeys the equation:
$$T ( t ) = {T}_{env} + ( T ( 0 ) - {T}_{env} ) {e}^{- rt} .$$

15. The system of claim 9, wherein the mitigation comprises at least one of modification to the power, a level of the aspiration, and a level of the irrigation.

16. A computer-based surgical support method comprising executing computing code associated with a surgical console, which executing performs the steps of:
receiving aspiration data regarding aspiration;
receiving characteristics of a surgical procedure, including at least a power level of a surgical instrument associated with the surgery;
applying a model estimating temperature of the surgical instrument based collectively on the receiving steps; and
mitigating adverse patient effects if the temperature is estimated by the model as likely to cause injury.

17. The method of claim 16, wherein receiving characteristics of the surgery further includes characteristics of particles affected by the surgery.

18. The method of claim 16, wherein the model is in accordance with the equation:
$$T ( t ) = {T}_{env} + ( T ( 0 ) - {T}_{env} ) {e}^{- rt} .$$

19. The method of claim 16, wherein the mitigating comprises alerting a surgeon.

20. The method of claim 16, wherein the mitigating comprises at least one of modifying the power level or a level of the aspiration.
